(19) United States
(12) Patent Application Publication
(10) Pub. No.: US 2019/0338338 A1
(43) Pub. Date: Nov. 7, 2019

(54) NOVEL USES OF CATALYTIC PROTEIN

(71) Applicant: The University of Stavanger, Stavanger (NO)

(72) Inventor: Clemens FURNES, Stavanger (NO)

(21) Appl. No.: 16/474,289

(22) PCT Filed: Dec. 20, 2017
(86) PCT No.: PCT/EP2017/083849

§ 371 Date: Jun. 27, 2019

(30) Foreign Application Priority Data
Dec. 29, 2016 (SE) 1651748-4

(51) Int. Cl. C12Q 1/52; A23J 1/00; A23J 1/04; A23J 1/02; G01N 33/04; G01N 33/08

(52) U.S. Cl. CPC C12Q 1/52; A23J 1/002; A23J 1/004; G01N 2333/91085; A23J 1/02; G01N 33/04; G01N 33/08; A23J 1/04

(57) ABSTRACT

The present invention relates to a method of enriching or screening for one or more target molecules from a primary source, which method comprises to provide at least one peptidic ligand comprising at least one lysine (K) and immobilized to a solid support; contacting the ligand(s) with a primary source comprising at least one target molecule comprising glutamine (Q); allowing the formation of complexes between the ligand and the target molecule; and separating the complexes from the primary source. The target molecule(s) comprises glutamine, and step c is performed in the presence of a catalytic protein comprising transglutaminase (TG). The catalytic protein comprising transglutaminase (TG) may comprise transglutaminase originating from fish, such as Atlantic cod TG (AcTG), e.g. AcTG-1, and the primary source may include waste material from the fish or dairy industry.

### TECHNICAL FIELD

The present invention relates to methods for detecting one or more target molecules from biological liquids. The target molecules may be biologically active proteins and/or peptides. The invention also relates to kits and other products for use in the methods according to the invention.

### BACKGROUND

One of the biggest challenges we are facing currently is that we have exceeded the world's ability to provide useful biological materials at a sustainable scale. We have to learn to do more with less. In order to meet the dramatic increase in the world's population, it is crucial to maximize the utilization of raw materials, and industries are urgently seeking new technologies and applications in order to tackle these challenges. By increasing and improving the utilization of raw materials, we can build new value chains. Higher value products can be achieved for instance by converting left-over biomaterials through treatment with enzymes, which at the same time will contribute to a zero waste society.

In the fish industry, very large volumes of raw materials are not used for human consumption and out of that, almost a fourth is dumped directly at sea. The value of this residual material is huge. In the dairy industry, waste milk also known as foremilk arising e.g. from the cleaning of equipment either go down the drain or are used as animal feed. Foremilk contains a wide range of nutrients and health inducing components, such as proteins and bioactive peptides.

A potentially interesting group of proteins useful in improved use of biological materials is transglutaminase (TG), which is a family of enzymes that catalyse an acyl-transfer reaction between the carboxamide group of a protein- or peptide-bound glutamine and the amino group of a lysine residue, resulting in the formation of an isopeptide bond. In general, these enzymes catalyse this reaction efficiently, having inherently small recognition sequences, high specificity for their glutamine-containing substrates and wide tolerance for the structure of the lysine-containing substrates.

Transglutaminases have been suggested for binding of fish muscle. More specifically, Moreno et al (Moreno, Carballo and Borderias in Research article DOI: 10.1002/jsfa.3245: “Influence of alginate and microbial transglutaminase as binding ingredients on restructured fish muscle processed at low temperature”, 13 May 2008) relates to the use of alginate and transglutaminase as additives in cold gelification of minced hake (Merluccius capensis) muscle. Among other things, it was found that the presence of sodium caseinate in combination with microbial transglutaminase was important in helping to increase the work of penetration in fish gels induced at low temperature. Examination of the chemical properties of the muscle gels showed that sodium alginate did not establish covalent protein-protein bonds, while microbial transglutaminase dramatically increased the number of covalent bonds formed between adjacent muscle proteins.

Thus, thermostable fish gels of good quality were produced with alginate as well as transglutaminase at temperatures below 10° C.

Analyses of proteins are often hampered by the difficulty of isolating large quantities of purified proteins from a native source. Furthermore, the proteins are usually isolated by purification of biological samples on columns and the various purified fractions are then tested for specific bioactivity. The proteins are further identified by mass spectrometry (MS). This is a time-consuming approach, and the MS analysis is often complicated by the small amount of specific proteins in the purified samples. Therefore, there is a need in this area for novel techniques and approaches.

### SUMMARY OF THE INVENTION

The present invention relates to the use of novel uses of at catalytic proteins, such as in the conversion of biomass to higher value products and in the screening for naturally reactive substrate sequence for such a catalytic protein.

One objective of the invention is to provide products and methods useful in the enrichment of, or screening for, biologically active molecules, such as proteins and peptides.

Thus, the invention relates to a method as defined by claim 1, which e.g. may be used to recover valuable proteins from waste products in the fish and/or dairy industry.

The invention also relates to a kit for performing such enrichment or screening.

Further details and advantages of the present invention will appear from the dependent claims as well as from the detailed disclosure of the invention below.

### BRIEF DESCRIPTION OF DRAWINGS

FIG. 1 shows the SDS-PAGE analysis from large-scale production of AcTG1-1 in E. coli at 13° C.

FIG. 2 shows the crosslinking of casein upon AcTG-1 treatment.

FIG. 3 shows an overview of the novel technology for targeted mining of bioactive molecules (i.e. peptides and proteins).

FIG. 4 shows the crosslinking of fish raw materials by AcTG-1 treatment followed by enterokinase treatment. The samples were run on 20% gel, 150 V for 1 h and then stained with Coomassie Brilliant Blue. The numbers at the top indicate wells and the molecular weight and the standard is indicated in the left margin of the figure. The dotted squares were cut out of the gel and sent to MS analysis. The position of the squares are indicated by arrows. Lane 1: Magic Marker (10 ul); Lane 2: Magic Marker (1 ul); Lane 3: sample with AcTG-1 treatment; Lane 4: sample without AcTG-1 treatment.

FIG. 5 shows crosslinking of fish raw materials to FLAG conjugated magnetic beads by AcTG-1 treatment followed by enterokinase treatment. The samples were run on 20% gel, 150 V for 1 h and then stained with Coomassie Brilliant Blue. The numbers at the top indicate wells and the molecular weight and the standard is indicated in the left margin of the figure. The dotted squares were cut out of the gel and sent to MS analysis. The position of the squares is indicated by arrow. Lane 1: Magic Marker; Lane 2: sample with AcTG-1 treatment; Lane 3: sample without AcTG-1 treatment.

FIG. 6 shows the crosslinking of Bovine foremilk materials to FLAG conjugated magnetic beads by AcTG-1 treatment followed by enterokinase treatment.

### DETAILED DESCRIPTION OF THE INVENTION

The present invention relates to the enrichment or screening of one or more target molecules from biological liquids.

Thus, a first aspect of the invention is a method of enriching or screening for one or more target molecules from a primary source, which method comprises
a. Providing at least one peptidic ligand comprising at least one lysine (K) and immobilized to a solid support;
b. Contacting the ligand(s) with a primary source comprising at least one target molecule comprising glutamine (Q);
c. Allowing the formation of complexes between the ligand and the target molecule; and
d. Separating the complexes from the primary source, wherein said at least one target molecule comprises glutamine, and wherein step c is performed in the presence of a catalytic protein comprising transglutaminase.

In this context, it is understood that the term “molecule” includes proteins as well as peptides, as well as any other materials that include the appropriate chain of amino acids for this purpose. Thus, the target molecule(s) may be any molecule recognized by catalytic protein and capable of forming at least one covalent bond with the peptidic ligands.

The catalytic protein comprising transglutaminase used according to the invention may be produced recombinantly, e.g. by expression in bacteria, yeast or any other suitable system. The bacteria may e.g. be E. coli, or any other suitable conventionally used bacterial host. The catalytic protein is advantageously of an apparent molecular weight of about 80 kda, which corresponds to monomeric transglutaminase 1 from Atlantic cod (AcTG-1). The sequence for AcTG-1 has been published, and is available e.g. on National Center for Biotechnology Information (NCBI).

In one embodiment, the peptidic ligand comprises a detectable tag, such as a FLAG tag or any other tag suitable for the purposes of the invention.

The solid support used in the present method may comprise magnetic beads, and the separation of step (d) may utilize the well known principles of magnetic separation. Magnetic separation is a well-known method in the area of separation, and the skilled person can easily obtain materials from commercial sources in order to perform the method of the invention.

Thus, the solid support may be FLAG-conjugated magnetic beads.

The method of the invention may comprise a step (e) during which target molecule(s) are separated from the ligand. In one embodiment, such separation is performed enzymatically, using e.g. enterokinase.

The primary source may comprise liquid material including target molecules, such as bioactive proteins or peptides. In one embodiment, the primary source originates from the fish or dairy industry.

A second aspect of the invention is a kit for enriching or screening for one or more target molecules from a primary source, which kit comprises magnetic beads to which peptidic ligand comprising at least one lysine (K) has been immobilised, wherein said at least one target molecules comprises glutamine, wherein the catalytic protein transglutaminase allows formation of complexes between the ligand and the target molecule and wherein the peptidic ligand comprises a detectable and enzymatically removable tag.

In one embodiment of the kit according to the invention, at least one target protein is a bioactive protein.

A third aspect of the invention is a system to screen for naturally reactive substrate sequence(s) for AcTG-1 that could be transferable to other transglutaminase enzymes as well.

### DETAILED DESCRIPTION OF THE DRAWINGS

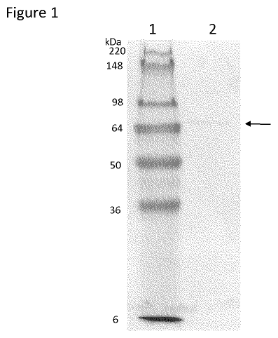

FIG. 1 shows the SDS-PAGE analysis from large-scale production of AcTG1-1 in E. coli at 13° C. Following harvesting of the protein extracts, the supernatant fraction was bound to the His-tag column, and was washed with 10 mM imidazole before elution with imidazole (lane 2). The fractions were run on a 12% SDS-PAGE, 180 V for 1 h and stained with Coomassie Brilliant Blue. The numbers at the top indicate lanes and the molecular weights of the standards are indicated in the left margin. Lane 1: Protein ladder (SeeBlue Plus2 Pre-Stained); Lane 2: Elution fraction. The position of the AcTG-1 is indicated by the arrow.

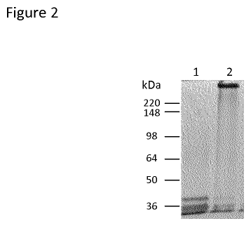

FIG. 2 shows the crosslinking of casein upon AcTG-1 treatment. Casein was incubated for 60 min in the presence of AcTG-1. Reactions were stopped by sample buffer addition and then analyzed on a 20% gel. Separated proteins are visualized in the gel by coomassie staining. Lane 1, O min; Lane 2, 60 min.

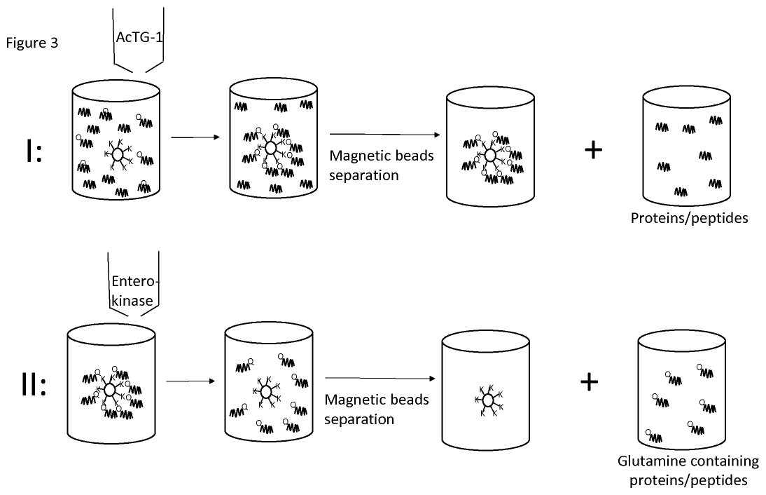

FIG. 3 shows an overview of the novel technology for targeted mining of bioactive molecules (i.e peptides and proteins). I) the solid support consists of FLAG-tag conjugated magnetic beads. To create a specific surface, displaying reactive lysine residues, to be cross-linked with glutamine residues in the target protein or peptides by AcTG-1 catalysis, a magnetic bead was coated with FLAG-tag. The FLAG-tag contains a lysine amino acid residue at the end of the sequence motif DYKDDDDK, allowing a covalent linkage between bioactive peptides and FLAG-tag. II). The FLAG-tag conjugated to magnetic beads can be removed from bioactive peptides and proteins once they have been isolated, by treatment with enterokinase that recognize the amino acid sequence DDDDK. This two-step isolation and enrichment procedure is expected to increase the sensitivity and efficiently of isolating bioactive peptides and proteins dramatically.

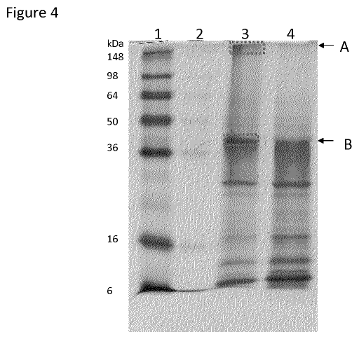

FIG. 4 shows the crosslinking of fish raw materials by AcTG-1 treatment followed by enterokinase treatment. The samples were run on 20% gel, 150 V for 1 h and then stained with Coomassie Brilliant Blue. The numbers at the top indicate wells and the molecular weight and the standard is indicated in the left margin of the figure. The dotted squares were cut out of the gel and sent to MS analysis. The position of the squares are indicated by arrows. Lane 1: Magic Marker (10 ul); Lane 2: Magic Marker (1 ul); Lane 3: sample with AcTG-1 treatment; Lane 4: sample without AcTG-1 treatment.

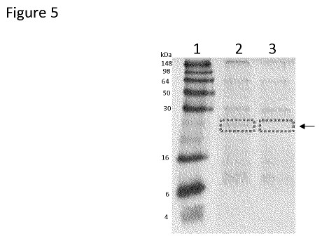

FIG. 5 shows crosslinking of fish raw materials to FLAG conjugated magnetic beads by AcTG-1 treatment followed by enterokinase treatment. The samples were run on 20% gel, 150 V for 1 h and then stained with Coomassie Brilliant Blue. The numbers at the top indicate wells and the molecular weight and the standard is indicated in the left margin of the figure. The dotted squares were cut out of the gel and sent to MS analysis. The position of the squares is indicated by arrow. Lane 1: Magic Marker; Lane 2: sample with AcTG-1 treatment; Lane 3: sample without AcTG-1 treatment.

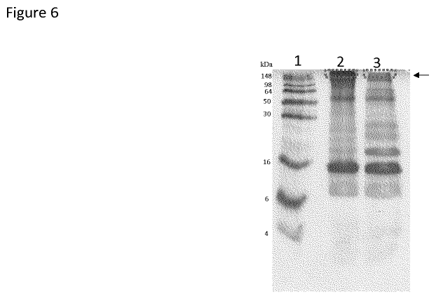

FIG. 6 shows the coupling of Bovine foremilk materials to FLAG conjugated magnetic beads by AcTG-1 treatment followed by enterokinase treatment. The samples were run on 20% gel, 150 V for 1 h and then stained with Coomassie Brilliant Blue. The numbers at the top indicate wells and the molecular weight and the standard is indicated in the left margin of the figure. The dotted squares were cut out of the gel and sent to MS analysis. The position of the squares is indicated by arrow. Lane 1: Magic Marker; Lane 2: sample with AcTG-1 treatment; Lane 3: sample without AcTG-1 treatment.

### EXPERIMENTAL PART

The present experiments are provided for illustrative purposes only, and should not be interpreted to limit the invention as defined by the appended claims.

### Example 1: Fishing for Bioactive Proteins—a Promising Tool for Enhanced Recovery of Proteins from Residual Materials

### Materials and Methods

### Construction of the Expression Plasmid of Atlantic Cod TG-1

Full-length AcTG-1 was cloned from the head kidney by a reverse-transcription polymerase chain reaction (RT-PCR) and rapid amplification of cDNA ends (RACE) [3]. A synthetic gene-encoding AcTG-1 with codon usage optimized for expression in E. coli flanked by restriction enzymes was ordered from Thermo Scientific. The region's encoded AcTG-1 gene were flanked by the restriction enzyme recognition sequence NdeI and SacI. The AcTG-1 fragment product generated by cleavage with NdeI and SacI restriction enzymes was excised from gel, and cloned into the NdeI and SacI digested pET151/D-TOPO vector (Invitrogen) to produce recombinant vector pET151/D-TOPO/AcTG-1. To confirm the fragment contained the AcTG-1 gene, sequencing with the T7 promoter/priming site 5′-TAATACGACTCACTATAGGG-3′ and the T7 reverse priming site 5′TAGTTATTGCTCAGCGGTGG-3′(universal primers) was conducted. A polyhistidine tag was present in AcTG-1 at the N-terminus, allowing the purification with His-Trap columns.

### Large-Scale Expression and Purification of His-Tag-rAcTG-1

Expression was performed using Escherichia coli BL21 (DE3) cells harboring petAcTG-1 (rAcTG-1) constructs grown in LB medium supplemented with 100 μg/ml ampicillin at 37° C. to an OD600 of 0.5-0.8. Recombinant protein expression was induced with 1 mM isopropyl β-D-1-thiogalactopyranoside (IPTG) at 13° C. for 16 h. The cells were harvested and lysed as described earlier. The filtered supernatant was applied onto a 1 ml His-Trap HP column (GE Healthcare). The column was washed with wash buffer (25 mM HEPES, 300 mM NaCl, 10 mM imidazole, pH 7.5), before rAcTG-1 was eluted using elution buffer (25 mM HEPES, 300 mM NaCl, 500 mM imidazole, pH 7.5). In all the following steps, fractions containing TG were determined by sodium dodecyl sulfate-polyacrylamide gel electrophoresis (SDS-PAGE), MS and immunoblotting.

### Electrophoresis and

The protein samples were analyzed by SDS-PAGE using 12% polyacrylamide gel following the method of Laemmli [10].

### Salmon and Bovine Residual Material

Salmon residual material was obtained using the method of Pampanin et al. 2016 (Daniela M. Pampanin, Marianne B. Haarr, Magne O. Sydnes—Natural peptides with antioxidant activity from Atlantic cod and Atlantic salmon residual material. Int. J. Appl. Res. Nat. Prod. (2016), 9 (2), 1-8.) and bovine waste milk was obtained from the dairy company “Q-meieriene”.

### Mass Spectrometry

The bands were excised by scalpel and analyzed by the proteomic facility at the University of Tromso. The protein samples were in-gel digested using trypsin and proteins were identified by quadrupole-time of flight (Q-TOF)/Liquid chromatography-mass spectrometry LC-MS.

### Protein Concentration

The amount of protein was determined with the BCA Protein assay kit (Thermo Scientific), using bovine serum albumin (BSA) as standard [11].

### Crosslinking of Casein by AcTG-1

Crosslinking of casein by AcTG-1 was detected by incubating 10 μL of enzyme extract and 10 μL of 1.0% casein at 16° C. for up to 1 h and then running the sample on SDS-PAGE.

### Labeling of Magnetic Beads

N-hydroxysuccinimide (NHS)-Activated magnetic beads were coupled to FLAG-tag manually with a magnetic stand according to the manual (Pierce). Briefly, 300 ul of beads were incubated with a solution of FLAG-tag peptides (2 mg/ml) for 2 h in 0.05 M sodium borate buffer with pH 8.5. Any remaining active NHS-ester groups were then quenched by incubation in 3 M ethanolamine at pH 9 for 2 h.

Fishing Bioactive Proteins and Peptides from Residual Materials

Following conjugation, 25 ul of prepared magnetic beads were incubated with a 5 ul extract (2 mg/ml) from Atlantic salmon (Salmon salar) or Bovine foermilk (2 mg/ml), 10 ul AcTG-1 (100 ug/ml)) and 5 ul 2× calcium buffer (10 mM CaCl₂), 3 mM DTT, 100 mM Tris-Hcl pH 7.5)), giving a final volume of 20 ul, for 1 h at 16° C. The beads were collected with a magnetic stand and then treated with 2 ul enterokinase (5 U/ul), 2 ul 10× reaction buffer and 16 ul deionized water at 25° C. for 16 h. The control was analyzed in parallel, where AcTG-1 was replaced with deionized water.

### Results

Recombinant expression of the construct pETAcTG-1 in E. coli BL21 cells at 13° C. showed expression of recombinant protein with a molecular weight of about 80 kDa upon protein purification and Coomassie staining after SDS-PAGE (FIG. 1, lane 2). The recombinant protein expressed in the soluble fraction was identified using MS (results not shown). The crosslinking activity of the enzyme was further studied, by incubation of casein with AcTG-1 for 1 h at 16° C. (FIG. 2). Electrophoresis of casein incubated with the enzyme extract showed that the intensity of casein decreased while that of crosslinked casein products with higher molecular weight increased (FIG. 2, lane 2).

Residual materials from both the fish and dairy were then used as starting material and AcTG-1 enzyme was used as a cross-linker to covalently immobilize peptides and proteins from raw materials on solid support (magnetic beads). The process was then followed by incubation with enterokinase, which mediated the release of the peptide or proteins of interest. Overview of the principle behind the method is shown (FIG. 3).

First, residual material from Atlantic salmon was tested by treating the samples with the AcTG-1 enzyme followed by enterokinase (FIG. 4). After the enzymatic reaction, the protein/peptide samples were run on a 20% SDS gel and two bands were digested enzymatically with trypsin and the resulting peptide mixture was analyzed by high-resolution MS. The five most frequent peptides from the two bands are shown in Table 1. This shows the presence peptide ranging in sizes from 7-21 amino acids and all ended in the amino acid lysine or arginine. No presence of amino acid glutamine was evident from these sequences. MS analysis revealed also the identities of a range of Atlantic salmon proteins, mostly muscles proteins. This test showed that the procedure did not interfere with the trypsin enzymatic digestion or with the MS analysis. The procedure was then repeated including the FLAG-conjugated magnetic beads. FIG. 5 shows one of three repeated results giving the same result with a band with approximately molecular size between 30 and 16 kDa. In order to differentiate between specifically and non-specifically bound molecules, the gel sample that had not been treated with TG was used as a control, with identical hits subtracted. The ten most frequent peptides after subtraction are shown in Table 2. They show variance in size from 7 to 19 amino acids. Furthermore, they all ended with lysine or arginine and seven of the peptides contain glutamine in their sequence.

Finally, we tested the procedure on bovine waste milk. On a SDS PAGE gel a more intense band with molecular size above 148 kDa was detected when treated with AcTG-1 (FIG. 6). This was repeated three times with same results. In order to differentiate between specifically and non-specifically bound molecules, the gel sample that had not been treated with AcTG-1 was used as a control, with identical hits subtracted. The most frequent peptide was DNPQTHYYAVAVVK (42 of total 79 peptides) and its identified protein was serotransferrin (Table 3).

Table 1 shows the most frequent peptide sequence found in the gel sample A and B crosslinked with AcTG-1 treatment followed by enterokinase treatment.

| Most frequent peptide | Most frequent peptide |
|---|---|
| (sample A) | (sample B) |
| INEMLDTK | GILAADESTGSVAK |
| AITDAAMMAEELKK | VIISAPSADAPMFVMGVNHEK |
| MEIDDLSSNMEAVAK | AISEELDNALNDMTSI |
| DLYANNVLSGGTTMYPGIADR | EITALAPSTMK |
| FSAEEMK | AVVLMSHLGRPDGNPMPDK |

Table 2 shows the most frequent peptide sequence found in the gel sample crosslinked to FLAG conjugated magnetic beads by AcTG-1 treatment followed by enterokinase treatment.

Most frequent peptide

MSADAMLAALLGTK
AITDAAMMAEELKK
LEEAGGATAAQIEMNK
DSTLIMQLLR
VAIQLNDTHPAMAIPELMR
IQLVEEELDR
YEVTTLR
TGGLMENFLVIHQLR
VDFDDIQK
LQGEVEDLMIDVER

Table 3 shows the most frequent peptide sequence and identified protein found in the gel sample crosslinked to FLAG conjugated magnetic beads by AcTG-1 treatment followed by enterokinase treatment.

| Most frequent peptide | Protein identified |
|---|---|
| DNPQTHYYAVAVVK | Serotransferrin |

1. A method of enriching or screening for one or more target molecules from a primary source, which method comprises
a) Providing at least one peptidic ligand comprising at least one lysine (K) and immobilized to a solid support;
b) Contacting the ligand(s) with a primary source comprising at least one target molecule comprising glutamine (Q);
c) Allowing the formation of complexes between the ligand and the target molecule; and
d) Separating the complexes from the primary source,
wherein said at least one target molecule comprises glutamine, and wherein step c is performed in the presence of a catalytic protein comprising transglutaminase.

2. A method according to claim 1, wherein the lysine-containing peptidic ligand is a peptide of 5-10 amino acids.

3. A method according to claim 2, wherein the amino acid sequence of the peptidic ligand comprises DYKDDDK or a His tag.

4. A method according to claim 1, wherein the solid support comprises a plurality of beads.

5. A method according to claim 1, wherein the solid support comprises a metal and the separation of step d is performed using magnetic separation.

6. A method according to claim 1, wherein the transglutaminase of step c comprises transglutaminase originating from fish, such as atlantic cod transglutaminase (AcTG), e.g. atlantic cod transglutaminase 1 (AcTG-1).

7. A method according to claim 1, which comprises a step (e) during which the target molecules are enzymatically separated from the peptidic ligands.

8. A method according to claim 1, wherein the primary source comprises material from the fish or dairy industry.

9. A method according to claim 1, wherein the glutamine-comprising target molecules are serotranferrin and lactoferrin.

10. A kit comprising magnetic beads to which at least one peptidic ligand comprising at least one lysine (K) has been immobilised; at least one transglutaminase capable of catalyzing the formation of complexes between the peptidic ligand and a target molecule which comprises at least one glutamine; in which kit the peptidic ligand comprises a detectable and enzymatically removable tag.

11. A kit according to claim 10, wherein at least one target protein is a bioactive protein.


SEQUENCE LISTING (23 sequences)

SEQ ID NO: 1 - protein, 8 aa, Artificial Sequence
  synthetic tag
DYKDDDDK

SEQ ID NO: 2 - protein, 6 aa, Artificial Sequence
  synthetic tag
HHHHHH

SEQ ID NO: 3 - protein, 8 aa, Artificial Sequence
  identified sequence
INEMLDTK

SEQ ID NO: 4 - protein, 14 aa, Artificial Sequence
  identified sequence
AITDAAMMAEELKK

SEQ ID NO: 5 - protein, 15 aa, Artificial Sequence
  identified sequence
MEIDDLSSNMEAVAK

SEQ ID NO: 6 - protein, 21 aa, Artificial Sequence
  identified sequence
DLYANNVLSGGTTMYPGIADR

SEQ ID NO: 7 - protein, 7 aa, Artificial Sequence
  identified sequence
FSAEEMK

SEQ ID NO: 8 - protein, 14 aa, Artificial Sequence
  identified sequence
GILAADESTGSVAK

SEQ ID NO: 9 - protein, 21 aa, Artificial Sequence
  identified sequence
VIISAPSADAPMFVMGVNHEK

SEQ ID NO: 10 - protein, 16 aa, Artificial Sequence
  identified sequence
AISEELDNALNDMTSI

SEQ ID NO: 11 - protein, 11 aa, Artificial Sequence
  identified sequence
EITALAPSTMK

SEQ ID NO: 12 - protein, 19 aa, Artificial Sequence
  identified sequence
AVVLMSHLGRPDGNPMPDK

SEQ ID NO: 13 - protein, 14 aa, Artificial Sequence
  identified sequence
MSADAMLAALLGTK

SEQ ID NO: 14 - protein, 14 aa, Artificial Sequence
  identified sequence
AITDAAMMAEELKK

SEQ ID NO: 15 - protein, 16 aa, Artificial Sequence
  identified sequence
LEEAGGATAAQIEMNK

SEQ ID NO: 16 - protein, 10 aa, Artificial Sequence
  identified sequence
DSTLIMQLLR

SEQ ID NO: 17 - protein, 19 aa, Artificial Sequence
  identified sequence
VAIQLNDTHPAMAIPELMR

SEQ ID NO: 18 - protein, 10 aa, Artificial Sequence
  identified sequence
IQLVEEELDR

SEQ ID NO: 19 - protein, 7 aa, Artificial Sequence
  identified sequence
YEVTTLR

SEQ ID NO: 20 - protein, 15 aa, Artificial Sequence
  identified sequence
TGGLMENFLVIHQLR

SEQ ID NO: 21 - protein, 8 aa, Artificial Sequence
  identified sequence
VDFDDIQK

SEQ ID NO: 22 - protein, 14 aa, Artificial Sequence
  identified sequence
LQGEVEDLMIDVER

SEQ ID NO: 23 - protein, 14 aa, Artificial Sequence
  identified sequence
DNPQTHYYAVAVVK